SEQUENCE LISTING (3 sequences)

SEQ ID NO: 1 - DNA, 47 bp, Artificial Sequence
  oligo hexamer
  misc_feature (1)..(1): 5′DBCO
  misc_feature (17)..(20): Nitroindol
  misc_feature (41)..(41): HEG Spacer
  misc_feature (42)..(43): n is a, c, g, or t
  misc_feature (44)..(47): n = a, c, g, or t; and wherein all the internucleotide linkages are phosphorothioate linkages
gcttgggtgtttaaccnnnnacttacgcgtgcgcaggccgnnnnnnn

SEQ ID NO: 2 - DNA, 24 bp, Saccharomyces cerevisiae
ccacaccacacccacacacccaca

SEQ ID NO: 3 - DNA, 23 bp, Saccharomyces cerevisiae
ggtgtgggtgtggtgtgtgtggg

(19) United States
(12) Patent Application Publication
(10) Pub. No.: US 2021/0198718 A1
(43) Pub. Date: Jul. 1, 2021

(54) METHOD OF ATTACHING ADAPTORS TO SINGLE-STRANDED REGIONS OF DOUBLE-STRANDED POLYNUCLEOTIDES

(71) Applicant: Oxford Nanopore Technologies Limited, Oxford (GB)

(72) Inventors: Daniel George Fordham, Oxford (GB); Phillip Laurence James, Oxford (GB)

(73) Assignee: Oxford Nanopore Technologies Limited, Oxford (GB)

(21) Appl. No.: 17/057,876

(22) PCT Filed: May 24, 2019
(86) PCT No.: PCT/GB2019/051442

§ 371 Date: Nov. 23, 2020

(30) Foreign Application Priority Data
May 24, 2018 (GB) 1808558.9

(51) Int. Cl. C12Q 1/6806; C12Q 1/6855

(52) U.S. Cl. CPC C12Q 1/6806; C12Q 1/6855

(57) ABSTRACT

A method of attaching an adapter to a polynucleotide comprising: providing a double stranded polynucleotide comprising a single stranded break point within its polynucleotide sequence; contacting said double stranded polynucleotide with an exonuclease to form a single stranded region initiated at said break point; attaching an adapter to said single stranded region.

### FIELD

The invention relates generally to methods of attaching adapters to polynucleotides. The attachment of adapters to polynucleotides prepares the polynucleotides for characterisation. The invention also relates generally to methods of characterising the adapted polynucleotides, and to reagents and kits for attaching adapters to polynucleotides and/or characterising the adapted polynucleotides.

### BACKGROUND

There are many commercial situations which require the preparation of a nucleic acid library. This is frequently achieved using a transposase. Depending on the transposase which is used to prepare the library it may be necessary to repair the transposition events in vitro before the library can be used, for example in sequencing.

There is currently a need for rapid and cheap polynucleotide (e.g. DNA or RNA) sequencing and identification technologies across a wide range of applications.

Transmembrane pores (nanopores) have great potential as direct, electrical biosensors for polymers and a variety of small molecules. In particular, recent focus has been given to nanopores as a potential DNA sequencing technology.

WO 2015/022544 discloses using a MuA transposase and a population of MuA substrates to produce a plurality of shorter, modified double stranded polynucleotides from a template double stranded polynucleotide.

### SUMMARY

The inventors have shown that sequencing can be initiated from single stranded break points in a polynucleotide. In particular, the inventors have shown that an exonuclease that can be initiated at a break point in a double stranded polynucleotide can be used to expose a single stranded region of polynucleotide. An adapter can then bind to the exposed single stranded region. The adapter can be a random adapter that hybridizes to any polynucleotide sequence (such as any DNA sequence), or a directed adapter that binds to a specific sequence or sequences. The 3′ end of the adapter may bind to the exposed single stranded region of the polynucleotide, leaving a free 5′ end. Once the 3′ end of the adapter is covalently attached to the 5′ end of the strand of polynucleotide adjacent to the exposed single stranded region, the free 5′ end of the adapter can be used as a primer site for amplification or as an attachment site for a sequencing adapter.

The new method has the advantage that it provides a fragmentation free method of preparing a sequencing sample. The method can be targeted to a particular region of a polynucleotide, or can be untargeted. The method is very quick and involves few steps. The method can be carried out without a clean-up step. All of these factors help aid the sequencing of long reads through fewer liquid handling steps. In one aspect, the method may be semi-directed and used to probe unknown regions of a polynucleotide starting from known sequence motifs, that can be conserved sequence motifs.

Accordingly, provided herein is a method of attaching an adapter to a polynucleotide comprising: providing a double stranded polynucleotide comprising a single stranded break point within its polynucleotide sequence; contacting said double stranded polynucleotide with an enzyme having exonuclease activity to form a single stranded region initiated at said break point; and attaching an adapter to said single stranded region. The adapter is typically attached by hybridising the adapter to said single stranded region and covalently attaching only the 3′ end of the adapter to the free 5′ end of a double stranded region of the polynucleotide adjacent to the single stranded region.

Also provided is a method of providing a double stranded polynucleotide comprising an exposed single stranded region flanked by double stranded regions; and attaching an adapter to said exposed stretch of single stranded polynucleotide.

The methods may further comprise covalently attaching the adapter to the double stranded polynucleotide and/or attaching a sequencing adapter or hybridizing a primer to the adapter.

Also provided are:
a method of characterizing a polynucleotide, comprising:
attaching an adapter to a polynucleotide by a method disclosed herein;
attaching a sequencing adapter to the adapter attached to the polynucleotide;
contacting the adapted polynucleotide with a nanopore such that the polynucleotide translocates through the nanopore; and
taking one or more measurements as the polynucleotide moves with respect to the nanopore, wherein the measurements are indicative of one or more characteristics of the polynucleotide and thereby characterising the polynucleotide;
a method of amplifying a polynucleotide, comprising:
attaching an adapter to a polynucleotide by a method disclosed herein;
hybridising a primer to the adapter attached to the polynucleotide;
carrying out an amplification reaction;
a kit comprising an enzyme having exonuclease activity and an adapter, wherein the adapter comprises a 5′ end and a 3′ end, wherein the 3′ end comprises a universal sequence of from 3 to 15 bases; and
A DNA comprising an adapter attached within a telomere, wherein the adapter comprises a 5′ end and a 3′ end, wherein the 3′ end is hybridized to the DNA.

### DESCRIPTION OF THE FIGURES

It is to be understood that Figures are for the illustration purposes and are not intended to be limiting.

FIG. 1 is a schematic illustration of how adapters can be attached to single stranded break points in a polynucleotide. A shows single stranded break points (1) in a polynucleotide, that may, for example, be random nicks in high molecular weight (HMW) DNA or targeted nicks (e.g. induced by Cas9). B shows how a 3′-5′ exonuclease (2) chews back from the break points, exposing single stranded DNA (ssDNA). C shows the hybridisation of intermediate adapters (3) to exposed ssDNA. In this Figure, a polymerase (4), which does not have strand displacement activity, extends the adapters in a 5′-3′ direction, although such extension is not required where the adapter attaches such that there is no gap between the 3′ end of the adapter and the 5′ end of the leading strand. D shows the attachment (5) of the 3′ end of the adapters to the 5′ end of the leading strand. Attachment may be achieved, for example, using a ligase or click chemistry.

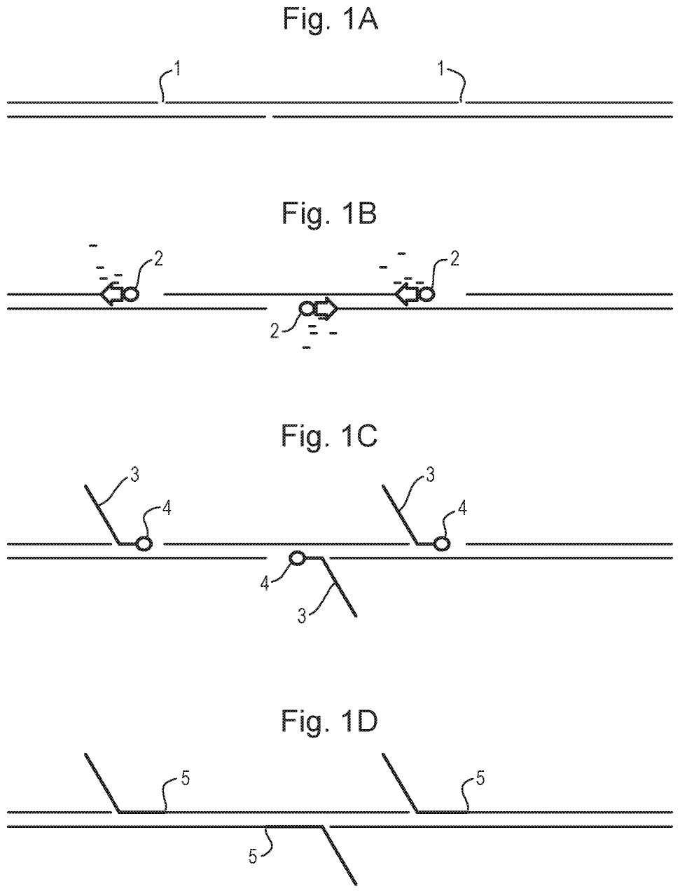

FIG. 2 is a Nx Plot showing the percentage of bases (x axis) from reads greater than N bases (y axis) in length in a sequencing library of E. coli genomic DNA having undergone adapter addition at nick sites using DNA polymerase I to open nicks and nick translate in the presence of dNTPs.

FIG. 3 shows the read length distribution of S. cervisiae sequences prepared using DNA polymerase I to open nicks in genomic DNA and DNA polymerase I with dNTPs and Sololobus Polymerase to nick translate and gap fill.

FIG. 4 is a schematic illustration of how adapters can be attached to a single stranded DNA region in a telomeric region of a chromosome. A depicts the structure of a telomere. B shows the DNA including the telomeric repeats (2) in linear form for clarity. The single stranded DNA overhang is at position (1). C shows the addition of an intermediate adapter with a hexamer sequence (3) of universal nucleotides (u) and a click group (4) which anneals to the single stranded DNA via hybridization of the universal nucleotides. Where hybridization leaves a gap between the 3′ end of the universal sequence in the adapter and the 5′ end of the telomere, a polymerase is used to fill the gap (for example, a polymerase with 5′-3′ exonuclease activity can extend into the 5′ end of the telomere).

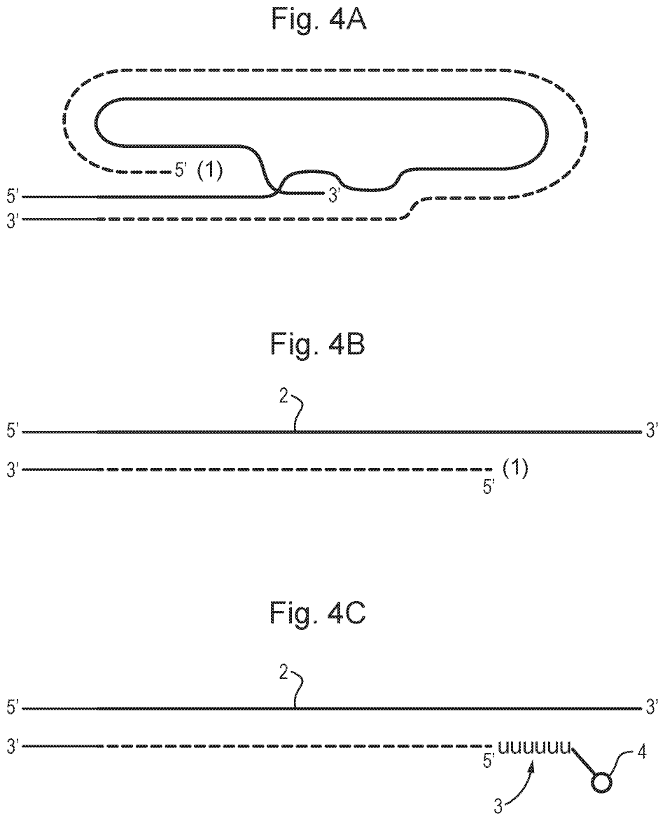

FIG. 5 is a DNA sequence alignment showing sequence alignment of sequences starting within telomeric regions of a S. cervisiae chromosome. The majority of reads are sequenced from the telomere towards the centromere.

FIG. 6 shows the proportional abundance of reads of given sequence lengths in comparison with Oxford Nanopore's SQL-LSK108 sequencing kit using E. coli genomic DNA as a template. Exonuclease III was used to open nicks and DNA polymerase I to nick translate from the 3′ end of a tailed random hexamer in the presence dNTPs. Duplicates of each preparation were used to generate the data.

FIG. 7 shows the results of an experiment to trial a number of different surfactants to try to “loosen” DNA to facilitate the interaction of enzymes and adapters with the centre of the DNA molecule. The surfactant Brij showed a modest increase in read length when compared with other surfactants and controls.

FIG. 8 shows the read statistics for a sequencing library prepared by adding adapters at nick sites in formamidopyrimidine [fapy]-DNA glycosylase (FPG) treated E. coli DNA (FPG introduces nicks at damaged bases) and E. coli DNA that has not been treated using FPG (no_fpg).

FIG. 9 shows the read lengths obtained when T7 DNA polymerase (a), Exonuclease III (b) and T4 DNA polymerase (c) were used chew back from a single nick site introduced into the 3221 bp plasmid pGEM®-11Zf(+) (Promega) using the nicking restriction endonuclease Nt.BspQI (NEB). T7 DNA polymerase provides the shortest reads, then Exonuclease III, with T4 DNA polymerase producing the longest reads.

FIG. 10 depicts the a pile up of the reads produced by the sequencing run after alignment or reads obtained by sequencing from a single nick introduced into DNA isolated from bacteriophage Lambda (NEB), which is 48,502 base pairs in length, using the nicking mutant nuclease variant of Cas9 (D10A) (NEB) after creating a stretch of single stranded bases (ssDNA) by chewing back from this nick site using Exonuclease III, hybridising an intermediate DNA adapter with complementarity to the target site, ligating the adapter onto the 5′ end of the exposed nick site and ligating the 5′ end of the intermediate adapter to a sequencing adapter. The reads can be seen to initiate from a single site at the 3′ end of the reference, demonstrating that sequencing can be targeted and target enriched using this method.

FIG. 11 is a schematic illustration of how adapters can be attached to single stranded break points in a polynucleotide. (1) shows single stranded break points in a polynucleotide, that may, for example, be random nicks in high molecular weight (HMW) DNA or targeted nicks (e.g. induced by Cas9). (2) shows an extended gap created by a 5′-3′ or a 3′-5′ exonuclease. (3) shows the hybridisation of an adapter to exposed ssDNA in the gap. (4) shows the binding of a polymerase to the 3′ end of the introduced adapter. (5) shows extension of the double stranded portion of the adapter using a polymerase which has 5′ exonuclease activity so that the native strand is displaced and digested. (6) shows the polymerase dissociating and the sealing of the breakpoint to covalently attach the adapter to the target polynucleotide. Attachment may be achieved, for example, using a ligase or click chemistry.

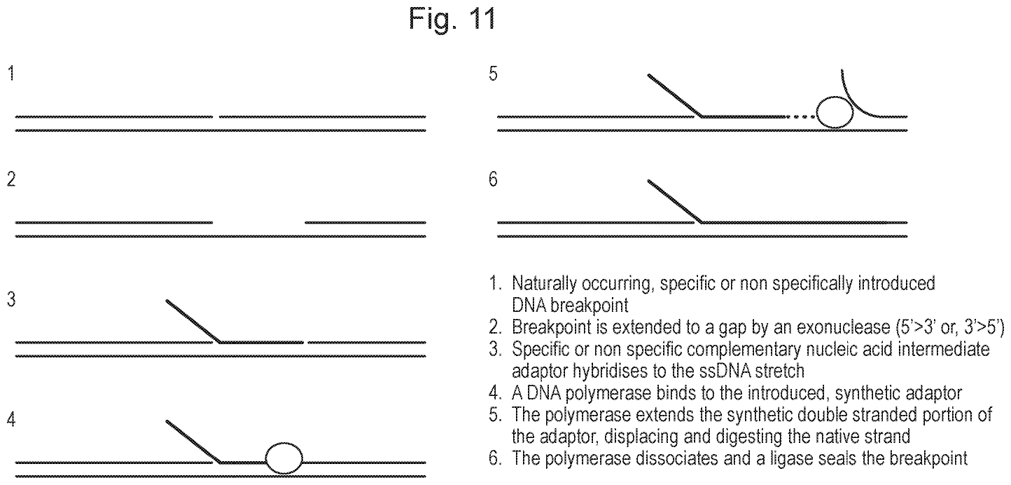

### DETAILED DESCRIPTION

It is to be understood that different applications of the disclosed methods and products may be tailored to the specific needs in the art. It is also to be understood that the terminology used herein is for the purpose of describing particular embodiments of the methods and products only, and is not intended to be limiting.

In addition as used in this specification and the appended claims, the singular forms “a”, “an”, and “the” include plural referents unless the content clearly dictates otherwise. Thus, for example, reference to “a polynucleotide” includes two or more polynucleotides, reference to “an anchor” refers to two or more anchors, reference to “a helicase” includes two or more helicases, and reference to “a transmembrane pore” includes two or more pores and the like.

All publications, patents and patent applications cited herein, whether supra or infra, are hereby incorporated by reference in their entirety.

### Method of Attaching Adapter

Provided herein is a method of attaching a sequencing adapter to a double stranded polynucleotide, the method comprising:
(a) providing a double stranded polynucleotide comprising a single stranded break point;
(b) contacting said double stranded polynucleotide with an exonuclease to form a single stranded region initiated from said break point; and
(c) attaching an adapter to the single stranded region.

The single stranded break point may be a nick in the polynucleotide, i.e. a break in the polynucleotide backbone, for example due to a missing phosphodiester bond between adjacent nucleotides in one strand of the double stranded polynucleotide. Thus, in one embodiment, the single stranded break point is a gap in the backbone of one strand of the double stranded polynucleotide.

The single stranded break point may be a gap in one strand of the double stranded polynucleotide. For example, 1, 2, 3, 4 or more nucleotides may be missing from one strand of the polynucleotide at the break point. The double stranded polynucleotide may be treated to facilitate access of an exonuclease to the break point. For example, the double stranded polynucleotide may be contacted with a surfactant such as Brij, Triton or Tween.

In one embodiment the break point occurs at the junction between a double stranded DNA and a telomere. For example, the break point may be the single stranded region at the junction between a double stranded DNA and a telomere.

The single stranded break point may be at a random position in the polynucleotide, or at a targeted position in the polynucleotide. The double stranded polynucleotide comprising a single stranded break point, may comprise multiple break points, such as from 2, 3, 4, 5, 6, 7, 8 or 9 to about 50, about 40, about 30, about 20 or about 10. Where the double stranded polynucleotide comprises multiple break points, at least one of the break points may be random and at least one of the break points may be at a targeted position. The multiple break points may comprise one or two break points at single stranded regions at telomeric sites and one or more break points along the length of the chromosome. The one or more break points along the length of the chromosome may be naturally occurring or artificially introduced.

The double stranded polynucleotide comprising at least one single stranded break point may be a polynucleotide that naturally comprises one or more single stranded break point. Accordingly, in one embodiment, the break point is naturally occurring. The double stranded polynucleotide may have at least one single stranded break point introduced artificially. The natural or artificial break point may arise as a result of DNA damage. A break point may be introduced at a random point in the double stranded polynucleotide, for example by mechanical force or by radiation. The mechanical force may, for example, be a shearing force as occurs when a double stranded polynucleotide is manipulated, such as by pipetting. A break point may be introduced a break point at a random position in a double stranded polynucleotide by an enzyme such as, for example, DNase 1, S1 nuclease or formamidopyrimidine [fapy]-DNA glycosylase (FPG).

A break point may be introduced at a targeted point in the double stranded polynucleotide. For example an enzyme may be used to introduce a single stranded break point at a targeted position in the polynucleotide. Examples of suitable enzymes include Cas9 nickase and nicking endonucleases.

The method may further comprise producing the double stranded polynucleotide comprising at least one single stranded break point. The double stranded polynucleotide comprising at least one single stranded break point may be produced by introducing single stranded break points by any of the means discussed above. For example, the double stranded polynucleotide comprising at least one single stranded break point within its polynucleotide sequence may be produced by contacting a double stranded polynucleotide with an enzyme that introduces a single stranded break point in the polynucleotide.

To introduce break points into a double stranded polynucleotide, the double stranded polynucleotide may be contacted with the enzyme for between about 10 seconds and about 1 hour, such as for 15, 20, 30, 40 or 50 seconds up to about 30 minutes, 20, minutes, 10 minutes, 5 minutes or 1 minute. The skilled person will be able to determine the exact time based on the enzyme used and, particularly where the enzyme introduces break points at random positions, the desired number of break points. Suitable reaction conditions for enzyme activity can readily be determined by the skilled person.

Also provided is a method of providing a double stranded polynucleotide comprising a single stranded polynucleotide region flanked by double stranded polynucleotide regions; and attaching an adapter to said exposed stretch of single stranded polynucleotide.

The single stranded polynucleotide region may be an exposed single stranded region that occurs naturally within a chromosome, such as the single stranded region at the terminus of a chromosome within a telomere.

The single stranded region is an exposed stretch of single stranded polynucleotide, typically flanked by double stranded polynucleotide regions. The single stranded region typically has a length of at least about 3 nucleotides, such as at least about 4, 5, 6, 7, 8, 9 or 10 nucleotides. The upper length of the single stranded region is not particularly limited. However, for characterisation methods, greater coverage of the polynucleotide is typically achieved where the length of the single stranded region is the same as or approximately the same as, such as 1, 2, 3, 4, 5 or 6 nucleotides longer than the region of the adapter being hybridized to the single stranded region. Thus, the single stranded region may typically have a length of up to about 6, 10, 15 or 20 nucleotides. The length of the single stranded region may be controlled by the reaction conditions used for step (b), such as the temperature and/or time for which the exonuclease is active. For example, the double stranded polynucleotide comprising at least one single stranded break point may contacted with the endonuclease for between about 10 seconds and about 1 hour, such as between about 15, 20 or 30 seconds and about 30 minutes, 20 minutes, 10 minutes, 5 minutes and 1 minute. It is within the routine skill for the skilled person to determine an appropriate time period.

In some embodiments, the exonuclease digestion may be carried out in the presence of a surfactant. Examples of suitable surfactants include Brji, Tween20 and TritonX-100.

The exonuclease is any enzyme having exonuclease activity. The exonuclease can act in either direction. The exonuclease may be a 3′-5′ exonuclease or a 5′-3′ exonuclease. The exonuclease may have both 3′-5′ exonuclease activity and 5′-3′ exonuclease activity. Preferably the exonuclease is a 3′-5′ exonuclease. Any exonuclease, or enzyme having exonuclease activity, can be used. Examples of suitable exonucleases include Exonuclease III, DNA polymerase I, T4 DNA polymerase and T7 DNA polymerase. Further examples of exonucleases include the following polymerases that have 3′-5′ exonuclease activity: Deep VentR™ DNA Polymerase, E. coli DNA Polymerase I, LongAmp® Taq DNA Polymerase, OneTaq®DNA Polymerase, phi29 DNA Polymerase, Phusion® High-Fidelity DNA Polymerase, Q5® DNA Polymerase, Taq polymerase, T4 DNA Polymerase, T7 DNA Polymerase and VentR® DNA Polymerase. Of these enzymes Taq polymerase and E. coli DNA Polymerase I have both 3′-5′ exonuclease activity and 5′-3′ exonuclease activity. The adapter is typically attached to the single stranded region by hybridization.

Methods are known in the art for repairing single stranded gaps in the double stranded constructs. For instance, the gaps can be repaired using a polymerase and a ligase, such as DNA polymerase and a DNA ligase. Alternatively, the gaps can be repaired using random oligonucleotides of sufficient length to bridge the gaps and a ligase.

A polymerase that acts in the 5′ to 3′ direction may be used to extend the end of the adapter after hybridisation of the adapter to the single stranded region to close the gap between the 3′ end of the adapter and the 5′ end of the flanking double stranded DNA. Suitable polymerases that act in the 5′ to 3′ direction include Taq polymerase (e.g. LongAmp® Taq DNA Polymerase), E. coli DNA polymerase I, Klenow fragment, Bst DNA polymerase (for example full length Bst DNA polymerase or large fragment Bst DNA polymerase), M-MuLV reverse transcriptase, phi29 polymerase, T4 DNA polymerase, T7 DNA polymerase, Vent and Deep Vent DNA polymerase (e.g. VentR® DNA Polymerase, VentR® (exo-) DNA Polymerase, Deep VentR™ DNA Polymerase, or Deep VentR™ (exo-) DNA Polymerase). Further examples of polymerases include Bsu DNA Polymerase (e.g. Large Fragment), Phusion® Hot StartFlex DNA Polymerase*, Phusion® High-FidelityDNA Polymerase*, Q5®+Q5® Hot Start DNA Polymerase, Sulfolobus DNA Polymerase IV and Therminator™ DNA Polymerase. In some embodiments, the polymerase may have 5′ exonuclease activity which destroys the leading strand (e.g. Bst DNA polymerase, E. coli DNA polymerase I, Taq polymerase (e.g. LongAmp® Taq DNA Polymerase)). In some embodiments, the polymerase does not have strand displacement activity (e.g. T4 DNA polymerase or Sulfolobus DNA Polymerase IV), allowing ligation to the 5′ end of the leading strand.

The method may further comprise covalently attaching the adapter to the double stranded polynucleotide. Typically the 3′ terminal nucleotide of the adapter is covalently attached to the 5′ terminal nucleotide adjacent to the single stranded region. The covalent attachment may be achieved by any suitable means, for example by ligation or click chemistry.

Thus, the method may further comprise covalently attaching, for example ligating the adapter to the double stranded polynucleotide. For example, a ligase, such as for example T4 DNA ligase, may be added to the sample to ligate the adapter to the double stranded polynucleotide. The adapter may be ligated to the double stranded polynucleotide in the absence of ATP or using gamma-S-ATP (ATPyS) instead of ATP. Examples of ligases that can be used include T4 DNA ligase, E. coli DNA ligase, Taq DNA ligase, Tma DNA ligase and 9° N DNA ligase. The adapter may be attached using a topoisomerisase. The topoisomerase may, for example be a member of any of the Moiety Classification (EC) groups 5.99.1.2 and 5.99.1.3.

Also provided is a method for preparing a double stranded polynucleotide for sequencing, the method comprising:

(a) providing a double stranded polynucleotide comprising a single stranded break point;

(b) contacting said double stranded polynucleotide with an enzyme having exonuclease activity to form a single stranded region initiated from said break point: and

(c) (i) hybridising a sequencing adapter comprising a single stranded portion to the single stranded region and covalently attaching the 3′ end of the sequencing adapter to the exposed 5′ end of the top strand of double stranded region adjacent to the single stranded region or;
(ii) hybridising an intermediate adapter to the single stranded region and covalently attaching the 3′ end of the intermediate adaptor to the exposed 5′ end of the top strand of the double stranded region adjacent to the single stranded region, and covalently attaching a sequencing adapter to the intermediate adapter. The sequencing adapter may be attached to the intermediate adapter by hybridising a single stranded region of the sequencing adapter to a 5′ portion of the intermediate adapter and covalently attaching the sequencing adapter to the intermediate adapter. Preferably the 3′ end of the sequencing adapter, or the 3′ end of a top strand of the sequencing adapter is covalently attached to the 5′ end of the intermediate adapter.

Step (c) of the method may further comprise filling the gap between the 3′ end of the sequencing adapter or intermediate adapter and 5′ end of the top strand of the double stranded region prior to covalent attachment. This may be achieved, for example, using a polymerase.

The method for attaching an adapter to a breakpoint within a double stranded polynucleotide produces polynucleotides for further manipulation and characterisation. The polynucleotides produced by the method typically comprise a 5′ terminal adapter ligated to target polynucleotide. For example, provided herein is a DNA molecule comprising an adapter attached within a telomere, wherein the adapter comprises a 5′ end and a 3′ end, wherein the 3′ end is hybridized to the DNA.

### Adapter

The adapter typically comprise a 3′ portion, or region, and a 5′ portion, or region. The 3′ portion of the adapter comprises a 3′ stretch of single stranded polynucleotide that hybridises to the exposed stretch of single stranded polynucleotide in the double stranded polynucleotide.

The 3′ stretch of single stranded polynucleotide in the adapter may be from about 3 to about 15 nucleotides or more in length, such as from about 4, 5, 6 or 7 to about 12, 10 or 8 nucleotides in length.

In one embodiment, the 3′ stretch of single stranded polynucleotide in the adapter comprises universal nucleotides that can hybridise to any polynucleotide sequence in the exposed stretch of single stranded polynucleotide in the double stranded polynucleotide. This is typically the case where the breakpoint is at a random location within the double stranded polynucleotide.

In one embodiment, the 3′ stretch of single stranded polynucleotide in the adapter comprises a sequence that is at least about 80%, such as at least about 90% or 95%, complementary to a polynucleotide sequence in the exposed stretch of single stranded polynucleotide in the double stranded polynucleotide. For example, the 3′ stretch of single stranded polynucleotide in the adapter may comprise a sequence that is exactly complementary to a polynucleotide sequence in the exposed stretch of single stranded polynucleotide in the double stranded polynucleotide. This may be the case where the breakpoint is at a targeted location within the double stranded polynucleotide. However, an adapter comprising a universal sequence in the 3′ portion may be used when the breakpoint is at a targeted location within the double stranded polynucleotide.

In one embodiment, the 3′ stretch of single stranded polynucleotide in the adapter hybridises to the exposed stretch of single stranded polynucleotide in the double stranded polynucleotide such that nucleotide at the 3′ terminus of the 3′ portion of the adapter hybridises to the nucleotide at the 5′ end of the exposed stretch of single stranded polynucleotide in the double stranded polynucleotide. This results in the 3′ end of the adapter abutting the 5′ end of the top strand of a double stranded region of the target polynucleotide. The 3′ end of the adapter can then be ligated directly to the 5′ end of the top strand of the target polynucleotide.

The 3′ stretch of single stranded polynucleotide in the adapter may be the same length as the exposed stretch of single stranded polynucleotide in the double stranded polynucleotide, or the 3′ stretch of single stranded polynucleotide in the adapter may be shorter than the length as the exposed stretch of single stranded polynucleotide in the double stranded polynucleotide.

The 5′ portion of the adapter does not hybridise to the exposed stretch of single stranded polynucleotide in the double stranded polynucleotide. The 5′ portion may be double stranded or single stranded. Typically the 5′ portion is single stranded or comprises a single stranded region. The single stranded region in the 5′ portion of the adapter may, for example, be used to attach the adapter to a further polypeptide, such as a sequencing, or other, adapter, or a primer. The 5′ portion may be designed to facilitate sequencing of the target polynucleotide. For example, the adapter that hybridises to the single stranded stretch in the target polynucleotide may be a sequencing adapter, such as a sequencing adapter for nanopore sequencing, or may be an intermediate adapter for attachment of a sequencing adapter.

The 5′ portion may have a length of, for example, from about 3 to about 45 nucleotides, such as about 6, 8, 10 or 15 to about 30, 25 or 20 nucleotides. The single stranded region of the 5′ portion, which may be all of the 5 portion, is typically at least about 3, 6, 8, 10 or 15 nucleotides in length.

The adapter typically has a length of from about 10 to about 50 nucleotides, such as from about 15 to about 40 or about 20 to about 30 nucleotides.

The adapter is typically a polynucleotide and may comprise DNA, RNA, modified DNA (such as a basic DNA), RNA, PNA, LNA, BNA and/or PEG. The adapter preferably comprises single stranded and/or double stranded DNA and/or RNA.

The adapter may further comprise a chemical group (e.g. click chemistry) for attachment of the 5′ portion of the adapter to a further adapter and/or a chemical group (e.g. click chemistry) for attachment of the 3′ portion of the adapter to the double stranded polynucleotide. Thus, the adapter may comprise a chemical group at the 5′ end or 3′ end. The chemical group at the 3′ end may be the same as the chemical group at the 5′ end, but is preferably different.

The adapter may further comprise a reactive group in the 3′ portion and/or in the 5′ portion. The reactive group in the 3′ portion may be used to covalently attach the adapter to the double stranded polynucleotide and/or the reactive group in the 5′ portion may be used to covalently attach the adapter to a further adapter. The reactive group at the 5′ end may be the same as, or different to, the reactive group at the 5′ end.

The reactive group may be used to ligate the fragments to the overhangs using click chemistry. Click chemistry is a term first introduced by Kolb et al. in 2001 to describe an expanding set of powerful, selective, and modular building blocks that work reliably in both small- and large-scale applications (Kolb H C, Finn, M G, Sharpless K B, Click chemistry: diverse chemical function from a few good reactions, Angew. Chem. Int. Ed. 40 (2001) 2004-2021). They have defined the set of stringent criteria for click chemistry as follows: “The reaction must be modular, wide in scope, give very high yields, generate only inoffensive by-products that can be removed by non-chromatographic methods, and be stereospecific (but not necessarily enantioselective). The required process characteristics include simple reaction conditions (ideally, the process should be insensitive to oxygen and water), readily available starting materials and reagents, the use of no solvent or a solvent that is benign (such as water) or easily removed, and simple product isolation. Purification if required must be by non-chromatographic methods, such as crystallization or distillation, and the product must be stable under physiological conditions”.

Suitable examples of click chemistry include, but are not limited to, the following:
(a) copper-free variant of the 1,3 dipolar cycloaddition reaction, where an azide reacts with an alkyne under strain, for example in a cyclooctane ring;
(b) the reaction of an oxygen nucleophile on one linker with an epoxide or aziridine reactive moiety on the other; and
(c) the Staudinger ligation, where the alkyne moiety can be replaced by an aryl phosphine, resulting in a specific reaction with the azide to give an amide bond.

Any reactive group may be used in the invention. The reactive group may be one that is suitable for click chemistry. The reactive group may be any of those disclosed in WO 2010/086602, particularly in Table 4 of that application. One particular example of a reactive group for click chemistry is DBCO (Dibenzoryclooctyl), which is commercially available.

In one embodiment, the adapter attached to the double stranded polynucleotide may be a sequencing adapter. The sequencing adapter may be ligated to the double stranded polynucleotide. The adapter may be ligated to the double stranded polynucleotide in the absence of ATP or using gamma-S-ATP (ATPyS) instead of ATP. It is preferred that the adapter is ligated to the polynucleotide in the absence of ATP where the adapter is a sequencing adapter to which a nucleic acid handling enzyme is bound. In this embodiment, the sequencing adapter may comprise a single stranded portion that hybridises to a stretch of single stranded polynucleotide in the double stranded polynucleotide.

Sequencing adapters are known in the art. The sequencing adapter may be altered for use in the present invention by adding on a single stranded region for hybridising to the exposed single stranded stretch in the target polynucleotide. The single stranded region is typically at the end of the 3′ end of one strand of the adapter. The sequencing adapter may be a Y adapter as described below.

### Adding Sequencing Adapter

In one embodiment, the method further comprises attaching a sequencing adapter to the 5′ portion of the adapter. Hence the adapter may act as a first, or intermediate, adapter. The sequencing adapter may comprise a single stranded portion that hybridises to a stretch of single stranded polynucleotide in the 5′ portion of the first adapter.

After hybridisation, the sequencing adapter may be covalently attached to the adapter using a ligase or by click chemistry. The ligase may, for example, be T4 DNA ligase, E. coli DNA ligase, Taq DNA ligase, Tma DNA ligase and 9° N DNA ligase. The adapter may be attached using a topoisomerisase. The topoisomerase may, for example be a member of any of the Moiety Classification (EC) groups 5.99.1.2 and 5.99.1.3.

The sequencing adapter may comprise a single stranded portion that hybridises to all or part of the 5′ region of the adapter that is attached to the target polynucleotide, i.e. the intermediate adapter. In this embodiment, the sequencing adapter is typically hybridised to the intermediate adapter prior to covalent attachment.

The sequencing adapter may be attached to the adapter after the adapter has been attached to the double stranded polynucleotide. Hence the method may comprise a step of attaching an adapter, typically by hybridisation and ligation, to an exposed stretch of single stranded polynucleotide in the double stranded polynucleotide and a sequential step of attaching the sequencing adapter to the adapter. Thus, the (intermediate) adapter may be added to the sample prior to adding the sequencing adapter to the sample.

The sequencing adapter may be attached to the adapter before the adapter is attached to the double stranded polynucleotide. Also, the method may comprise attaching an adapter to the exposed stretch of single stranded polynucleotide in the double stranded polynucleotide and attaching a sequencing adapter to a 5′ stretch of single stranded polynucleotide in the adapter in a single step. Thus, the sequencing adapter and the (intermediate) adapter may be added to the sample at the same time.

### Adding Primer

In one embodiment, the method further comprises amplifying a portion of the double stranded polynucleotide using at least one primer that hybridises to the 5′ portion of the adapter. The primer may hybridise to all or part of the 5′ portion of the adapter. Methods for designing suitable primers are well known in the art. The region of the double stranded polynucleotide that is attached to the adapter may then be amplified, for example by an isothermal amplification method. Suitable amplification reactions, such as isothermal amplification methods, are known in the art.

Thus, provided herein is a method of amplifying a polynucleotide, comprising attaching an adapter to a polynucleotide by a method disclosed herein; hybridising a primer to the adapter attached to the polynucleotide; and carrying out an amplification reaction.

### Kit

Also provided is a kit for attaching an adapter to a breakpoint within a double stranded polynucleotide. The kit comprises an enzyme having exonuclease activity and an adapter, wherein the adapter comprises a 3′ portion and a 5′ portion and the 3′ portion comprises a universal sequence of from 3 to 15 nucleotides. The universal sequence is capable of hybridising to any single stranded polynucleotide sequence. The adapter may have any of the features described above.

The kit may further comprise a polymerase and/or a ligase.

The kit may further comprise a means for introducing break points into a double stranded polynucleotide. For example, the kit may comprise an enzyme that introduces random break points in a double stranded polynucleotide, such as, for example, DNase I, S1 nuclease and/or FPG. For example, the kit may comprise an enzyme that introduces targeted break points in a double stranded polynucleotide, such as, for example, a Cas9 nickase and/or a nicking endonuclease.

The kit may further comprise a surfactant, such as Brij, Triton or Tween.

The kit may further comprise a sequencing adapter. The sequencing adapter is capable of hybridizing to the 5′ portion of the first adapter. The sequencing adapter may comprises a single stranded portion that has a sequence that is complementary to, or has a sequence that is at least 80% identical to the complement of, a single stranded region of the 5′ portion of the first adapter. In one embodiment the sequencing adapter may be a Y-adapter. Y adapters for nanopore sequencing are described in the art. The Y-adapter typically comprises a 5′ leader sequence that includes a single stranded polynucleotide, a double stranded region and, on the opposite strand to the leader sequence, a 3′ single stranded region to which a membrane tether is, or can be, attached. Membrane tethers are described in the art and can be, for example, a lipid, fatty acid, sterol, carbon nanotube, polypeptide, protein or amino acid, for example cholesterol, palmitate or tocopherol. The membrane tether may comprise thiol, biotin or a surfactant. Suitable membrane tethers and methods of attaching membrane tethers to adapters are disclosed in WO 2012/164270. The membrane tether may be attached to the single stranded region directly, or may be attached to a polynucleotide that hybridizes to a portion of the single stranded region. A nucleic acid handling polynucleotide, typically a helicase, may be pre-bound to the 5′ leader sequence in the Y-adapter and stalled at a spacer as disclosed in WO 2014/135838.

In addition, the Y adapter comprises a single stranded region at the 5′ end of the opposite strand to the leader sequence that is capable of hybridizing to the (intermediate) adapter that is attached to the double stranded polynucleotide. This single stranded region at the 5′ end of the opposite strand to the leader sequence, may for example, have a length of from about 3 to about 15 nucleotides, such as about 6, 8, 10 or 12 nucleotides.

The kit may comprise a primer. The primer may have a sequence that is complementary to, or a sequence that is at least 80% identical to the complement of, the 5′ region of the adapter.

The kit of the invention may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotides, a membrane as defined above or voltage or patch clamp apparatus. Reagents may be present in the kit in a dry state such that a fluid sample resuspends the reagents. The kit may also, optionally, comprise instructions to enable the kit to be used in the method of attaching an adapter to a breakpoint within a double stranded polynucleotide or details regarding which patients the method may be used for. The kit may, optionally, comprise nucleotides.

### Characterisation Method

A method of characterizing a polynucleotide, comprising: attaching an adapter to a polynucleotide by a method disclosed herein; attaching a sequencing adapter to the adapter attached to the polynucleotide; contacting the adapted polynucleotide with a nanopore such that the polynucleotide translocates through the nanopore; and taking one or more measurements as the polynucleotide moves with respect to the nanopore, wherein the measurements are indicative of one or more characteristics of the polynucleotide and thereby characterising the polynucleotide.

Any number of polynucleotides can be investigated. For instance, the method of the invention may concern characterising two or more polynucleotides, such as 3 or more, 4 or more, 5 or more, 6 or more, 7 or more, 8 or more, 9 or more, 10 or more, 20 or more, 30 or more, 50 or more, 100 or more, 500 or more, or 1,000 or more polynucleotides. The polynucleotides can be naturally occurring or artificial.

The method may involve measuring two, three, four or five or more characteristics of each polynucleotide. The one or more characteristics are preferably selected from (i) the length of the polynucleotide, (ii) the identity of the polynucleotide, (iii) the sequence of the polynucleotide, (iv) the secondary structure of the polynucleotide and (v) whether or not the polynucleotide is modified.

The methods may be carried out using any sequencing apparatus, including for example, an apparatus that comprises a nanopore, such as a transmembrane protein pore or a solid state pore. For example, the apparatus may comprise a chamber comprising an aqueous solution that is separated into a cis section and a trans section. The skilled person is readily able to choose a suitable apparatus from those available in the art.

Also provided is a method of sequencing a polynucleotide comprising attaching an adapter to a polynucleotide by a method disclosed herein, either wherein the adapter is a sequencing adapter, or wherein the adapter is an intermediate adapter and the method further comprises attaching a sequencing adapter to the intermediate adapter, and sequencing the polynucleotide.

Any method may be used for sequencing. Typically a next generation sequencing method is used, for example, any method of ensemble or single molecule sequencing. Examples of suitable sequencing methods include standard sequencing by synthesis (SBS) sequencing methods, such as Genia, PacBio, Illumina, Helicos, Solid or 454 methods, and single molecule sequencing methods, which may be either direct or indirect, these can be performed using a nanopore, such as using Oxford Nanopore Technologies' sequencing technology, or via any other known method, such as AFM, Sequencing by Hybridisation or Stratos' Sequencing by Expansion. Sequencing adapters for use in these methods are known in the art.

### Sample

The sample may be any suitable sample comprising polynucleotides. The polynucleotides may, for example, comprise the products of a PCR reaction, genomic DNA, the products of a endonuclease digestion and/or a DNA library.

The sample may be a biological sample. The invention may be carried out in vitro on a sample obtained from or extracted from any organism or microorganism. The organism or microorganism is typically archaean, prokaryotic or eukaryotic and typically belongs to one the five kingdoms: plantae, animalia, fungi, monera and protista. The invention may be carried out in vitro on a sample obtained from or extracted from any virus.

The sample is preferably a fluid sample. The sample typically comprises a body fluid. The body fluid may be obtained from a human or animal. The human or animal may have, be suspected of having or be at risk of a disease. The sample may be urine, lymph, saliva, mucus, seminal fluid or amniotic fluid, but is preferably whole blood, plasma or serum. Typically, the sample is human in origin, but alternatively it may be from another mammal such as from commercially farmed animals such as horses, cattle, sheep or pigs or may alternatively be pets such as cats or dogs.

Alternatively a sample of plant origin is typically obtained from a commercial crop, such as a cereal, legume, fruit or vegetable, for example wheat, barley, oats, canola, maize, soya, rice, bananas, apples, tomatoes, potatoes, grapes, tobacco, beans, lentils, sugar cane, cocoa, cotton, tea or coffee.

The sample may be a non-biological sample. The non-biological sample is preferably a fluid sample. Examples of non-biological samples include surgical fluids, water such as drinking water, sea water or river water, and reagents for laboratory tests. The sample may be processed prior to carrying out the method, for example by centrifugation or by passage through a membrane that filters out unwanted molecules or cells, such as red blood cells. The method may be performed on the sample immediately upon being taken. The sample may also be typically stored prior to the method, preferably below −70° C.

The sample may comprise genomic DNA. The genomic DNA may be fragmented. Preferably the genomic DNA is not fragmented. The genomic DNA may be from any organism. The genomic DNA may be human genomic DNA.

### Universal Nucleotides

A universal nucleotide is one which will hybridise to some degree to all of the nucleotides in the template polynucleotide. A universal nucleotide is preferably one which will hybridise to some degree to nucleotides comprising the nucleosides adenosine (A), thymine (T), uracil (U), guanine (G) and cytosine (C). A universal nucleotide may hybridise more strongly to some nucleotides than to others. For instance, a universal nucleotide (I) comprising the nucleoside, 2′-deoxyinosine, will show a preferential order of pairing of I-C>I-A>I-G approximately =I-T. It is only necessary that the universal nucleotides used in the adapter hybridise to all of the nucleotides in the double stranded polynucleotide. For example, when the double stranded polynucleotide is DNA, the universal nucleotides in the adapter need only bind to A, C, G and T.

A universal nucleotide may comprise one of the following nucleobases: hypoxanthine, 4-nitroindole, 5-nitroindole, 6-nitroindole, 3-nitropyrrole, nitroimidazole, 4-nitropyrazole, 4-nitrobenzimidazole, 5-nitroindazole, 4-aminobenzimidazole or phenyl (C6-aromatic ring. The universal nucleotide more preferably comprises one of the following nucleosides: 2′-deoxyinosine, inosine, 7-deaza-2′-deoxyinosine, 7-deaza-inosine, 2-aza-deoxyinosine, 2-aza-inosine, 4-nitroindole 2′-deoxyribonucleoside, 4-nitroindole ribonucleoside, 5-nitroindole 2′-deoxyribonucleoside, 5-nitroindole ribonucleoside, 6-nitroindole 2′-deoxyribonucleoside, 6-nitroindole ribonucleoside, 3-nitropyrrole 2′-deoxyribonucleoside, 3-nitropyrrole ribonucleoside, an acyclic sugar analogue of hypoxanthine, nitroimidazole 2′-deoxyribonucleoside, nitroimidazole ribonucleoside, 4-nitropyrazole 2′-deoxyribonucleoside, 4-nitropyrazole ribonucleoside, 4-nitrobenzimidazole 2′-deoxyribonucleoside, 4-nitrobenzimidazole ribonucleoside, 5-nitroindazole 2′-deoxyribonucleoside, 5-nitroindazole ribonucleoside, 4-aminobenzimidazole 2′-deoxyribonucleoside, 4-aminobenzimidazole ribonucleoside, phenyl C-ribonucleoside or phenyl C-2′-deoxyribosyl nucleoside.

The following Examples illustrate the method.

### Example 1: Attaching an Adapter to Natural Nicks in E. coli Genomic DNA Using a Single Enzyme (DNA Polymerase I) Method

Eight parallel 25 μl reactions were set up containing 1 μg of genomic DNA with 10 units of DNA polymerase I (New England Biolabs) in 1×NEB buffer 2, 0.05% Brij surfactant (Sigma-Aldrich) and incubated at room temperature for 10 minutes. Heat denatured oligo hexamer (5′-2GCTTGGGTGTTTAACC5555ACTTACGCGTGCGCAGGCCG6NNN*N*N*N-3′—wherein 2=DBCO (click chemistry reactive group), 5=Nitroindol (universal base), 6=HEG (spacer) and *=phosphorothioate linkage) with rapid attachment chemistry (ATD Bio) were added to a final concentration of 68 μM along with dNTPs (New England Biolabs) to a final concentration of 68 mM. Samples were incubated at room temperature for 10 minutes prior to the addition of 5 μl of NebNext Quick ligase, 10 μl of 5× NebNext Quick ligase buffer (New England Biolabs) and made up to 50 μl with nuclease free water. Samples were incubated at room temperature for 15 minutes then 20 units of Exonuclease I (New England Biolabs) were added to each reaction and incubated at room temp for 10 minutes. A 0.4× Agencourt Ampure XP (Beckman Coulter) bead clean up with 1×70% ethanol wash and 1×109 wash buffer (Oxford Nanopore Technologies) was performed and DNA was eluted from beads in 80 μl of buffer comprising of 100 mM NaCl and 20 mM Tris pH 8 at room temperature for 10 minutes. Individual reactions were pooled and heat denatured at 85° C. for 3 minutes before snap cooling on ice for 2 minutes. 40 μl of RAP adapter (Oxford Nanopore Technologies) was added and incubated with gentle mixing for 20 minutes at room temperature. A 0.4× Agencourt Ampure XP bead clean-up was performed according to the manufacturer's instructions but ethanol washes were replaced with 109 wash buffer (Oxford Nanopore Technologies). DNA was eluted in 24.75 109 elution buffer and combined with 37.5 μl SQB, 11.75 LLB and 1 μl SQT (Oxford Nanopore Technologies) prior to loading on a 9.4.1 Oxford Nanopore Flowcell 1 according to the manufacturer's instructions.

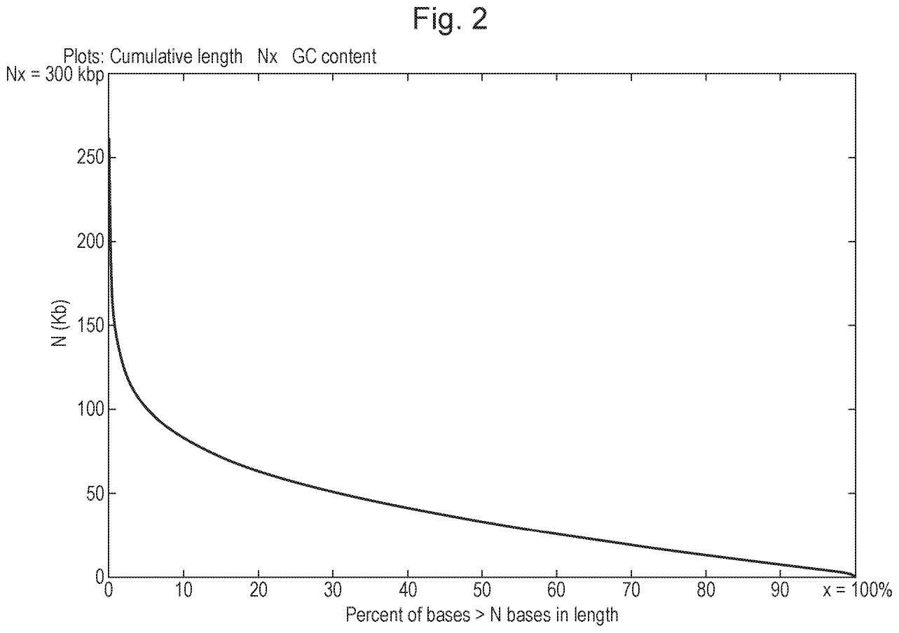

The lengths of the sequenced polynucleotides are shown in FIG. 2.

### Example 2: Attaching an Adapter to Natural Nicks in S. cervisiae Genomic DNA Using a Single Enzyme (DNA Polymerase I) Method with Gap Filling (Sulfolobus DNA Polymerase IV)

Eight parallel 25 μl reactions were set up containing 1 μg of genomic DNA with 10 units of DNA polymerase I (New England Biolabs) in 1×NEB buffer 2, 0.05% Brij surfactant (Sigma-Aldrich) and incubated at room temperature for 10 minutes. Heat denatured oligo hexamer (5′-2GCTTGGGTGTTTAACC5555ACTTACGCGTGCGCAGGCCG6NNN*N*N*N-3′—where 2=DBCO, 5=Nitroindol, 6=HEG and *=phosphorothioate linkage) with rapid attachment chemistry (ATD Bio) were added to a final concentration of 68 μM along with dNTPs (New England Biolabs) to a final concentration of 68 mM with 4 units of Sulfolobus DNA polymerase IV (New England Biolabs). Samples were incubated at room temperature for 10 minutes prior to the addition of 5 μl of NebNext Quick ligase, 10 μl of 5× NebNext Quick ligase buffer (New England Biolabs) and made up to 50 μl with nuclease free water. Samples were incubated at room temperature for 15 minutes then 20 units of Exonuclease I (New England Biolabs) were added to each reaction and incubated at room temp for 10 minutes. A 0.4× Agencourt Ampure XP (Beckman Coulter) bead clean up with 1×70% ethanol wash and 1×109 wash buffer (Oxford Nanopore Technologies) was performed and DNA was eluted from beads in 80 μl of buffer comprising of 100 mM NaCl and 20 mM Tris pH 8 at room temperature for 10 minutes. Individual reactions were pooled and heat denatured at 85° C. for 3 minutes before snap cooling on ice for 2 minutes. 40 μl of RAP adapter (Oxford Nanopore Technologies) was added and incubated with gentle mixing for 20 minutes at room temperature. A 0.4× Agencourt Ampure XP bead clean-up was performed according to the manufacturer's instructions but ethanol washes were replaced with 109 wash buffer (Oxford Nanopore Technologies). DNA was eluted in 24.75 109 elution buffer and combined with 37.5 μl SQB, 11.75 LLB and 1 μl SQT (Oxford Nanopore Technologies) prior to loading on a 9.4.1 Oxford Nanopore Flowcell 1 according to the manufacturer's instructions.

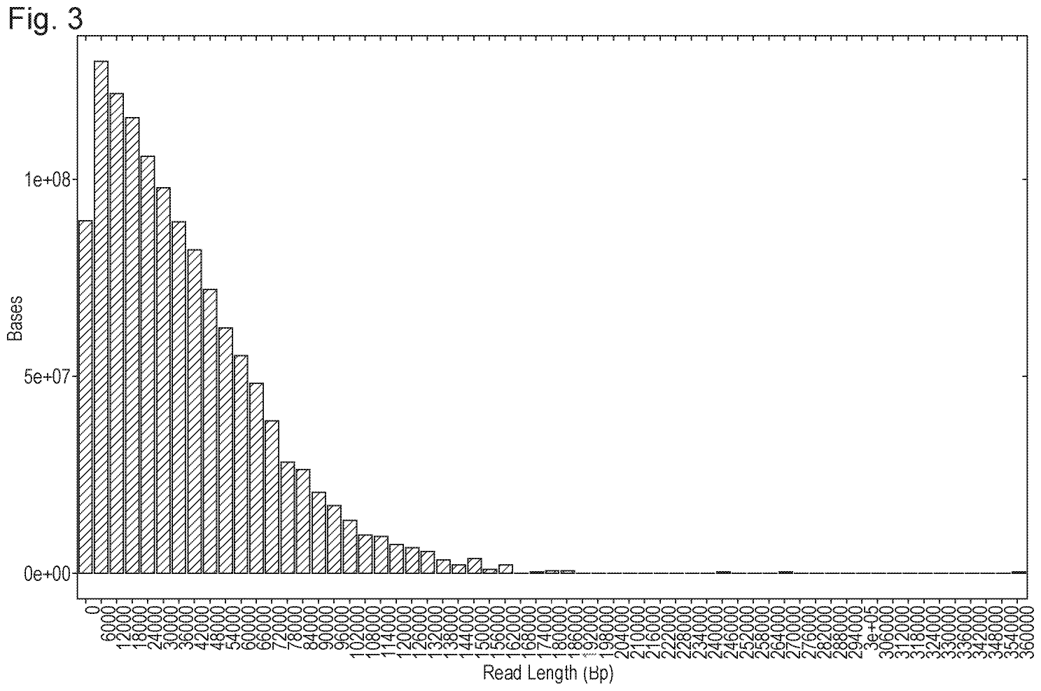

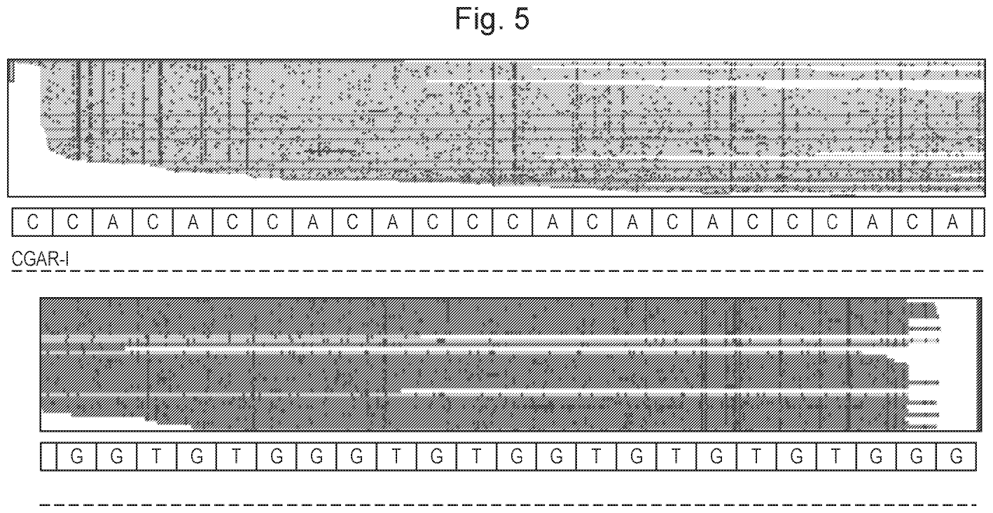

The lengths of the sequenced polynucleotides are shown in FIG. 3. FIG. 5 shows that some of the reads start within telomeric regions of the chromosomes.

### Example 3: Attaching an Adapter to Natural Nicks in E. coli Genomic DNA Using a Double Enzyme (DNA Polymerase I and Exonuclease III) Method

Eight parallel 25 μl reactions were set up containing 1 μg of genomic DNA with 100 units of Exonuclease III (New England Biolabs) in 1×NEB buffer 2, 0.05% Brij surfactant (Sigma-Aldrich) and incubated at room temperature for 45 seconds. Reactions were halted by the addition of ETDA (Sigma-Aldrich) to a final concentration of 5 mM. Exonuclease III was then heat inactivated at 70° C. for 20 minutes. Heat denatured oligo hexamer (5′-2GCTTGGGTGTTTAACC5555ACTTACGCGTGCGCAGGCCG6NNN*N*N*N-3′—where 2=DBCO, 5=Nitroindol, 6=HEG and *=phosphorothioate linkage) with rapid attachment chemistry (ATD Bio) were added to a final concentration of 68 μM along with dNTPs (New England Biolabs) to a final concentration of 68 mM with 10 units of DNA polymerase I (New England Biolabs). Samples were incubated at room temperature for 10 minutes prior to the addition of 5 μl of NebNext Quick ligase, 10 μl of 5× NebNext Quick ligase buffer (New England Biolabs) and made up to 50 μl with nuclease free water. Samples were incubated at room temperature for 15 minutes then 20 units of Exonuclease I (New England Biolabs) were added to each reaction and incubated at room temp for 10 minutes. A 0.4× Agencourt Ampure XP (Beckman Coulter) bead clean up with 1×70% ethanol wash and 1×109 wash buffer (Oxford Nanopore Technologies) was performed and DNA was eluted from beads in 80 μl of buffer comprising of 100 mM NaCl and 20 mM Tris pH 8 at room temperature for 10 minutes. Individual reactions were pooled and heat denatured at 85° C. for 3 minutes before snap cooling on ice for 2 minutes. 40 μl of RAP adapter (Oxford Nanopore Technologies) was added and incubated with gentle mixing for 20 minutes at room temperature. A 0.4× Agencourt Ampure XP bead cleanup was performed according to the manufacturer's instructions but ethanol washes were replaced with 109 wash buffer (Oxford Nanopore Technologies). DNA was eluted in 24.75 109 elution buffer and combined with 37.5 μl SQB, 11.75 LLB and 1 μl SQT (Oxford Nanopore Technologies) prior to loading on a 9.4.1 Oxford Nanopore Flowcell 1 according to the manufacturer's instructions.

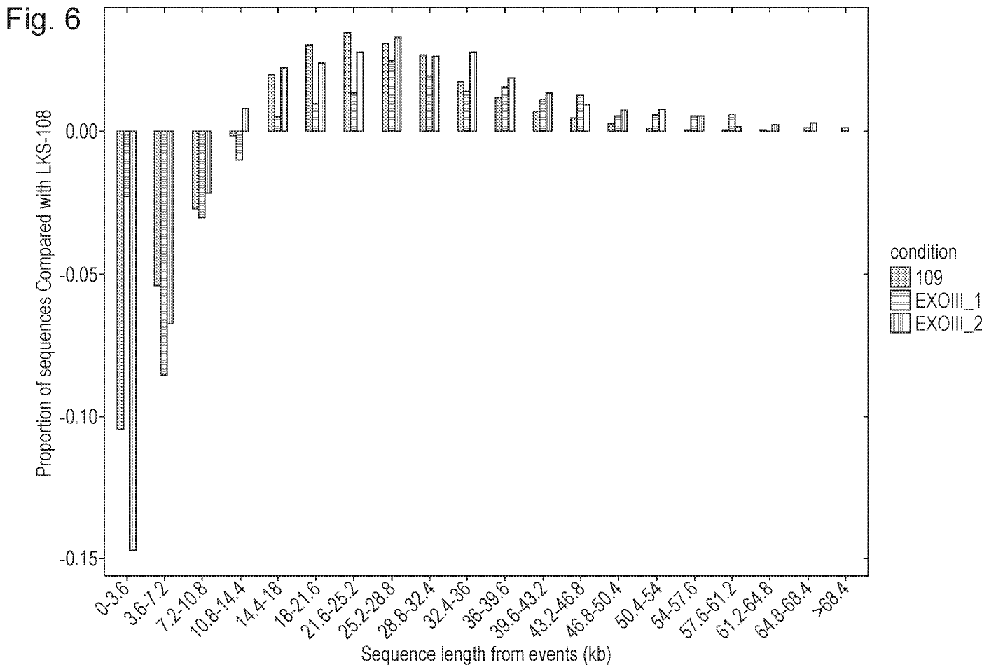

FIG. 6 shows the proportional abundance of reads of different sequence lengths in comparison to the number of reads of the same lengths obtained using the commercially available SQK-LSK108 sequencing kit (Oxford Nanopore Technologies). The number of shorter reads is reduced and the number of longer reads is increased relative to the prior art method when using the present method.

### Example 4: Use of Surfactants

The method of Example 3 was performed as described above in the presence of the surfactant Brij (3)(Sigma-Aldrich), Tween20 (Sigma-Aldrich), Triton X-100 (Sigma-Aldrich) or nuclease free water throughout. Each surfactant had a final concentration of 0.05%.

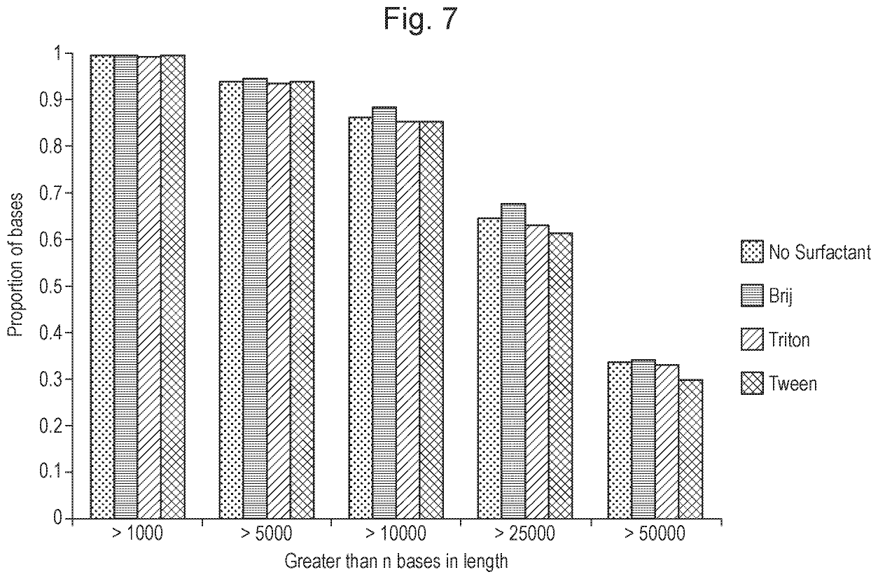

The results are shown in FIG. 7. The surfactant Brij showed a modest increase in read length when compared with other surfactants and controls.

### Example 5: Attaching an Adapter to Induced Nicks in E. coli Genomic DNA

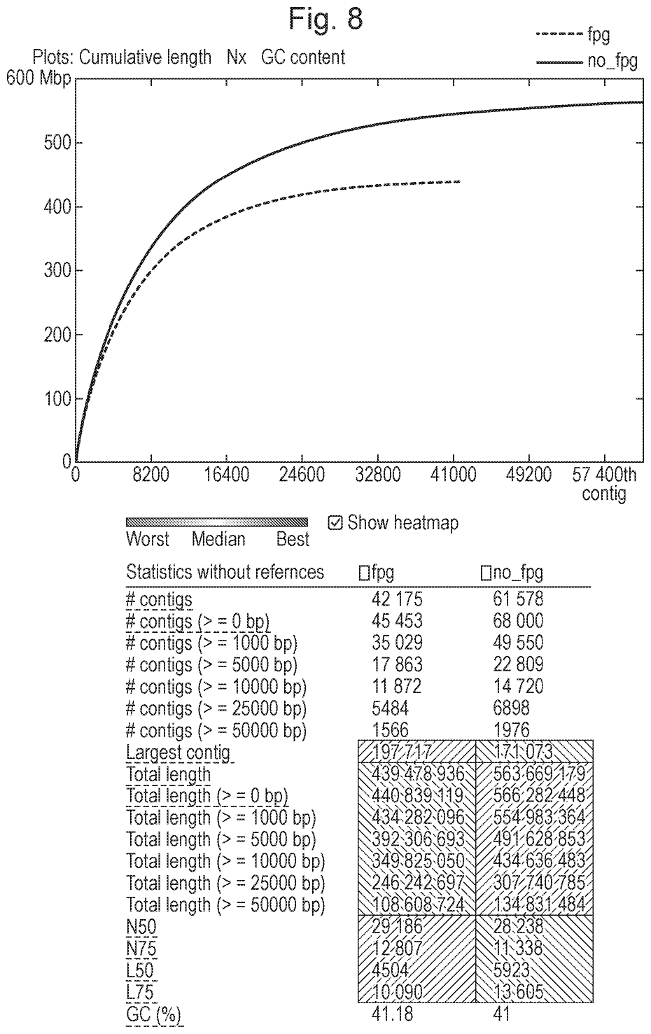

The method of Example 1 was repeated using formamidopyrimidine [fapy]-DNA glycosylase (FPG) treated E. coli DNA (FPG introduces nicks at damaged bases) and the results were compared to when a sequencing library was prepared using untreated E. coli DNA. The results are shown in FIG. 8. The read lengths are reduced when the library is prepared using the FPG-treated DNA in which the number of nicks is greater.

### Example 6: Attaching an Adapter to Nicks Introduced Using a Restriction Nickase

A single nick was introduced into the 3221 bp plasmid pGEM®-11Zf(+) (Promega) using the nicking restriction endonuclease Nt.BspQI (NEB). The nicking endonuclease cuts the phosphate backbone of DNA on one strand one base downstream of the recognition site (GCTCTTCN\hat{ }). A stretch of single stranded bases (ssDNA) was created by chewing back from this nick site using a 3′-5′ exonuclease.

In this experiment the exonuclease activities of 3 different exonucleases were compared: Exonuclease III (NEB), T4 DNA polymerase (NEB), and T7 DNA polymerase (NEB). After the ssDNA stretches were exposed, an intermediate DNA adapter with complementarity to the known site was hybridised and ligated onto the 5′ end of the exposed nick site. At the 5′ end of the intermediate adapter there is an overhang site specific to a sequencing adapter. The products of this ligation were then sequenced using an Oxford Nanopore Technologies Ltd. MinION and the read lengths compared.

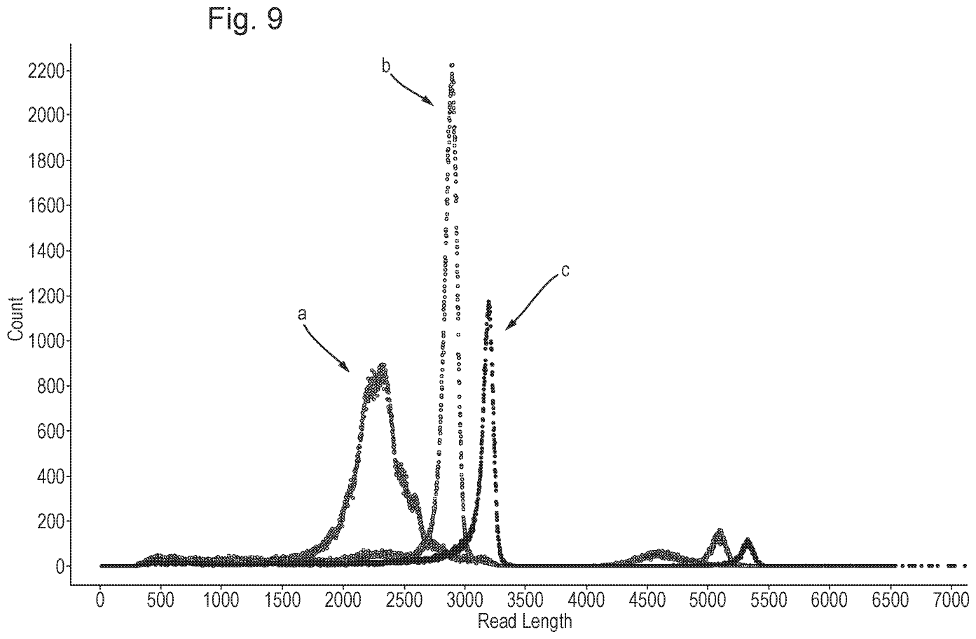

FIG. 9 depicts the read lengths of the three experiments with T7 DNA polymerase providing the shortest reads, then Exonuclease III, with T4 DNA polymerase producing the longest reads. The read lengths can be used to infer the exonuclease activity of the three enzymes as the reads can only initiate from the point of the nick induced by Nt.BspQI and alignment to a reference sequence confirms this.

### Example 7: Attaching an Adapter to Nicks Introduced Using a Restriction Nickase

A single nick was introduced into the DNA isolated from bacteriophage Lambda (NEB), which is 48,502 base pairs in length, using the nicking mutant nuclease variant of Cas9 (D10A) (NEB). The nicking enzyme specifically cuts the phosphate backbone of DNA on opposite strand using a guide RNA complementary to a site with a 3′ NGG protospacer adjacent motif (PAM). Once the nick was induced, a stretch of single stranded bases (ssDNA) was created by chewing back from this nick site using a 3′-5′ exonuclease.

In this experiment Exonuclease III (NEB) was used to produce the ssDNA target site. After the ssDNA stretch was exposed, an intermediate DNA adapter with complementarity to the known site was hybridised and ligated onto the 5′ end of the exposed nick site. At the 5′ end of the intermediate adapter there is an overhang site specific to a sequencing adapter, which was ligated to the intermediate adapter. The products of this ligation were then sequenced using an Oxford Nanopore Technologies Ltd. MinION and the reads aligned to the known reference for Lambda bacteriophage.

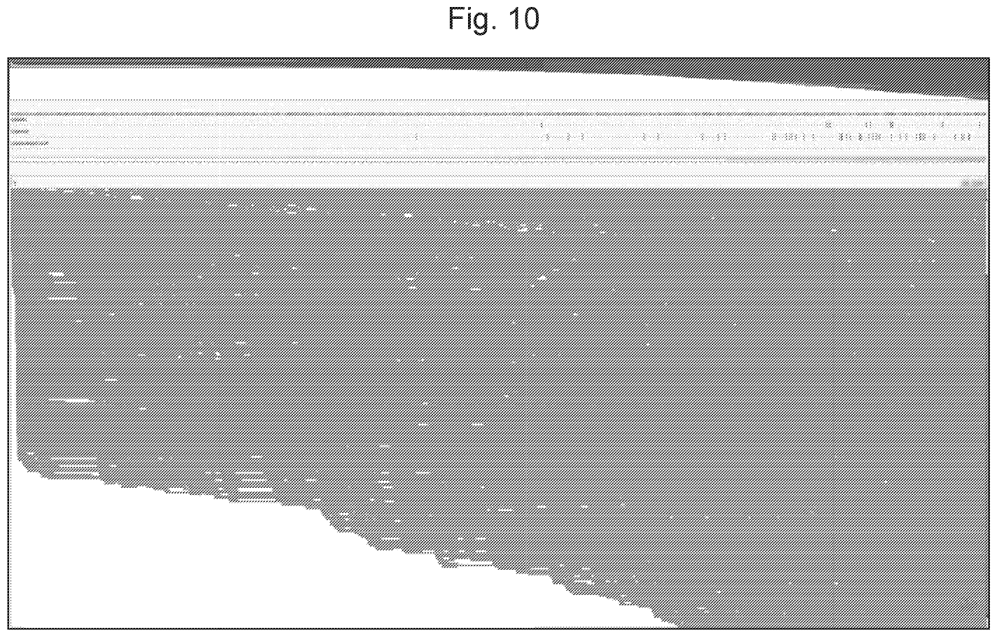

FIG. 10 depicts the a pile up of the reads produced by the sequencing run after alignment. The reads can be seen to initiate from a single site at the 3′ end of the reference, demonstrating that sequencing can be targeted and target enriched using this method.

1. A method of attaching an adapter to a polynucleotide comprising:
(a) providing a double stranded polynucleotide comprising a single stranded break point within its polynucleotide sequence;
(b) contacting said double stranded polynucleotide with an enzyme having exonuclease activity to form a single stranded region initiated at said break point;
(c) hybridising an adapter to said single stranded region;
(d) covalently attaching only the 3′ end of the adapter to the double stranded region of the polynucleotide adjacent to the single stranded region, wherein the 3′ end is covalently attached to the free 5′ end of the double stranded region.

2. A method according to claim 1, wherein the break point is naturally occurring, optionally wherein the break point is a break in the backbone of one strand of the double stranded polynucleotide.

3. (canceled)

4. A method according to claim 1, wherein the break point is a single stranded region within a telomere.

5. A method according to claim 1, wherein the break point is introduced by mechanical force or radiation.

6. A method according to claim 1, wherein the break point is introduced using an enzyme and the enzyme is selected from the group consisting of: DNase 1, S1 nuclease, Cas9 nickase or a nicking endonuclease.

7. A method according to claim 1, wherein the break point is at a random position in the polynucleotide or is located at a targeted position in the polynucleotide.

8.-10. (canceled)

11. A method according to claim 1, which further comprises initially producing the double stranded polynucleotide comprising at least one single stranded break point within its polynucleotide sequence.

12. A method according to claim 1, which further comprises initially producing the double stranded polynucleotide comprising at least one single stranded break point within its polynucleotide sequence and wherein the double stranded polynucleotide comprising at least one single stranded break point within its polynucleotide sequence is produced by contacting a double stranded polynucleotide with an enzyme that introduces a single stranded break point in the polynucleotide.

13. (canceled)

14. A method according to claim 1, wherein the exonuclease activity is 3′ to 5′ exonuclease activity.

15. A method according to claim 1, wherein the enzyme having exonuclease activity is Exonuclease III, DNA polymerase I, T4 DNA polymerase or T7 DNA polymerase.

16. (canceled)

17. A method according to claim 1, wherein the single stranded region is at least about 3 nucleotides in length.

18. (canceled)

19. A method according to claim 1, wherein the adapter comprises a 3′ stretch of single stranded polynucleotide that hybridises to the single stranded region in the double stranded polynucleotide, optionally wherein the 3′ stretch of single stranded polynucleotide in the adapter hybridises to the single stranded region in the double stranded polynucleotide such that base at the 3′ terminus of the 3′ stretch of single stranded polynucleotide in the adapter hybridises to the base in the double stranded polynucleotide at the 5′ end single stranded region.

20. (canceled)

21. A method according to claim 1, wherein the adapter comprises a 3′ stretch of single stranded polynucleotide that hybridises to the single stranded region in the double stranded polynucleotide, wherein (i) the 3′ stretch of single stranded polynucleotide in the adapter is the same length as the single stranded region in the double stranded polynucleotide; or(ii) the 3′ stretch of single stranded polynucleotide in the adapter is from about 3 to about 15 nucleotides in length; or (iii) the 3′ stretch of single stranded polynucleotide in the adapter is from about 5 to about 8 nucleotides in length; or(vi) the 3′ stretch of single stranded polynucleotide in the adapter comprises universal bases that can hybridise to any polynucleotide sequence in the single stranded region in the double stranded polynucleotide; or (vii) the 3′ stretch of single stranded polynucleotide in the adapter comprises universal bases that can hybridise to any polynucleotide sequence in the single stranded region in the double stranded polynucleotide; or (viii) the 3′ stretch of single stranded polynucleotide in the adapter comprises a sequence that is at least about 80% complementary to a polynucleotide sequence in the single stranded region in the double stranded polynucleotide; or (ix) the 3′ stretch of single stranded polynucleotide in the adapter comprises a sequence that is exactly complementary to a polynucleotide sequence in the single stranded region in the double stranded polynucleotide.

22.-26. (canceled)

27. A method according to claim 1, which further comprises covalently attaching the adapter to the double stranded polynucleotide, optionally wherein the adapter is covalently attached to the double stranded polynucleotide by ligation or click chemistry and wherein the 3′ end of the adapter is ligated to the 5′ terminal nucleotide adjacent to the single stranded region.

28.-30. (canceled)

31. A method according to claim 1, wherein the adapter comprises a 5′ stretch of single stranded polynucleotide that does not hybridise to the exposed stretch of single stranded polynucleotide in the double stranded polynucleotide.

32. A method according to claim 1, which further comprises attaching a sequencing adapter to the 5′ stretch of single stranded polynucleotide in the adapter, optionally wherein the sequencing adapter comprises a single stranded portion that hybridises to the 5′ stretch of single stranded polynucleotide in the adapter.

33.-37. (canceled)

38. A method of characterising a polynucleotide, comprising: attaching an adapter to a polynucleotide by providing a double stranded polynucleotide comprising a single stranded break point within its polynucleotide sequence; contacting said double stranded polynucleotide with an exonuclease to form a single stranded region initiated at said break point; attaching an adapter to said single stranded region; and attaching a sequencing adapter to the adapter attached to the polynucleotide; contacting the adapted polynucleotide with a nanopore such that the polynucleotide translocates through the nanopore; and taking one or more measurements as the polynucleotide moves with respect to the nanopore, wherein the measurements are indicative of one or more characteristics of the polynucleotide and thereby characterising the polynucleotide.

39. A method of amplifying a polynucleotide, comprising attaching an adapter to a polynucleotide by a method disclosed herein; hybridising a primer to the adapter attached to the polynucleotide; and carrying out an amplification reaction.

40.-42. (canceled)
